(19) United States
(12) Patent Application Publication
(10) Pub. No.: US 2003/0029446 A1
(43) Pub. Date: Feb. 13, 2003

(54) Misting device with a temperature-controlled air delivery system qa

(76) Inventors: John F. Skinner, Irving, TX (US); Brian D. Zelickson, Minneapolis, MN (US)

Correspondence Address: JENKENS & GILCHRIST, PC, 1445 ROSS AVENUE, SUITE 3200, DALLAS, TX, 75202, US

(21) Appl. No.: 10/058,856

(22) Filed: Jan. 28, 2002

Related U.S. Application Data
(63) Continuation-in-part of application No. 09/925,144, filed on Aug. 8, 2001.

(51) Int. Cl. A61M 11/00; F23D 11/00; F23D 14/00; A61M 16/00

(52) U.S. Cl. 128/200.14; 128/203.26

(57) ABSTRACT

A system for cooling and/or heating skin during laser surgery, the system utilizing cooled or heated gaseous fluid such as air containing a fragrance injected therein. The system comprises a fluid delivery apparatus having an air delivery system. The air delivery system has an air outlet and delivers air through the air outlet. A resilient delivery tube is connected to the air outlet, and terminates at a delivery outlet. A misting tube is provided having a fluid receiving end and a fluid delivering end. The fluid delivering end is connected to the resilient delivery tube at a point adjacent to the delivery outlet. A fluid source is provided connected to the misting tube at the fluid receiving end. Fluid from the fluid source may be fragranced, medicated, lubricated or otherwise treated. Fluid from the fluid source is combined in-stream in a misting pattern with air from the resilient delivery tube to provide the fluid at a predetermined location. In certain embodiments, a nozzle may be utilized to form the misting pattern. Likewise, air from the air delivery system may be cool and/or heated prior to combination with fluid from the fluid source. In operation, the air supply system delivers air through the resilient delivery tube. The fluid is then introduced in a misting pattern into the resilient delivery tube near the delivery outlet. The flow of air and fluid from said delivery tube is then directed onto a desired location.

### CROSS-REFERENCE

This application is a continuation-in-part of pending U.S. patent application Ser. No. 09/925,144 filed Aug. 8, 2001.

### BACKGROUND OF THE INVENTION

1. Field of the Invention

The present invention relates to methods of and apparatus for cooling and/or heating areas to which energy is applied and, more particularly, but not way of limitation, to means for the cooling and/or heating of an area of skin during the medical treatment thereof, including the introduction of a fragrance for masking odors typically associated therewith and soothing the skin.

2. History of Related Art

It is commonly understood that the heating of organic matter produces distinct odors. Conventional dermatological techniques that incorporate the heating of regions of the human skin likewise produce undesirable odors for which the utilization of a fragrance for masking said odors is deemed advantageous. In more recent years the utilization of laser technology for medical procedures, including dermatological treatment, has further accentuated the desirability of controlling odor associated with such procedures. Various innovations have addressed these concerns. Such innovations include controlling proportions of photomechanical and photothermal damage by selection of laser parameters, chromophore particle size and/or pulse duration, and infiltrating a photoactivated drug into hair ducts such as those disclosed in U.S. Pat. No. 6,050,990.

Notwithstanding the above referenced innovations and the use of lasers and light for dermatological treatment, the need still exists for improved methods of an apparatus for introducing fragrances and/or medication in a system that minimizes the utilization of extraneous materials during the medical procedure, and which may be implemented with a minimum of effort and maximum efficiency. Cost is likewise a consideration with a primary focus being the effectiveness for the patient and reliability for the physician. The present invention provides such a system by utilizing a method of and an apparatus for mixing a fragrance in air being used to cool and/or heat an area of skin during a dermatological procedure.

### SUMMARY OF INVENTION

The present invention relates to a method of and apparatus for cooling and/or heating skin areas during dermatological medical procedures. More particularly, one aspect of the present invention includes a system for cooling and/or heating skin during laser surgery, the system utilizing cool and/or heated air containing a fragrance injected therein. The system comprises a fluid delivery apparatus having an air delivery system. The air delivery system has an air outlet and delivers air through the air outlet. A resilient delivery tube is connected to the air outlet, and terminates at a delivery outlet.

A misting tube is provided having a fluid receiving end and a fluid delivering end. The fluid delivering end is connected to the resilient delivery tube at a point adjacent to the delivery outlet. A fluid source is provided connected to the misting tube at the fluid receiving end. Fluid from the fluid source may be fragranced, medicated, lubricated or otherwise treated. Fluid from the fluid source is combined in-stream in a misting pattern with air from the resilient delivery tube to provide the fluid at a predetermined location. In certain embodiments, a nozzle may be utilized to form the misting pattern. Likewise, air from the air delivery system may be cooled and/or heated prior to or after combination with fluid from the fluid source.

In operation, the air supply system delivers air through the resilient delivery tube. The fluid is then introduced in a misting pattern into the resilient delivery tube near the delivery outlet. The flow of air and fluid from said delivery tube is then directed onto a desired location.

### BRIEF DESCRIPTION OF THE DRAWINGS

A more complete understanding of the method and apparatus of the present invention may be obtained by reference to the following detailed description when taken in conjunction with the accompanying drawings wherein:

FIG. 1 is a diagrammatic view of the system according to the present invention,

FIG. 2 is an enlarged view of the delivery tube and fluid nozzle of FIG. 1; and

FIG. 3 is a diagrammatic view of an alternate embodiment for the system.

### DETAILED DESCRIPTION OF THE DRAWINGS

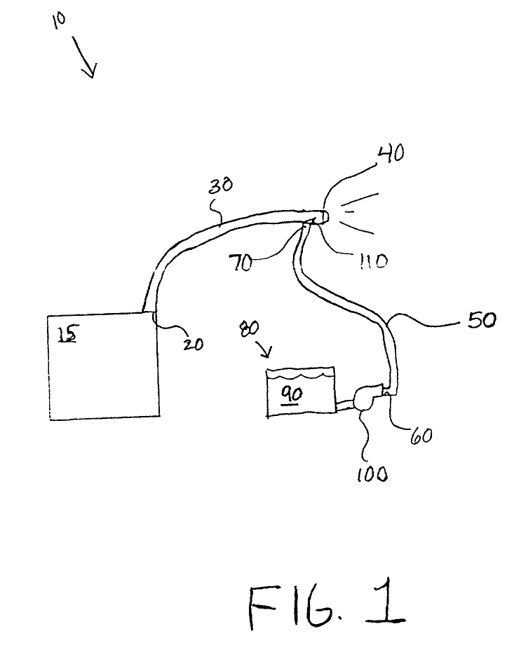

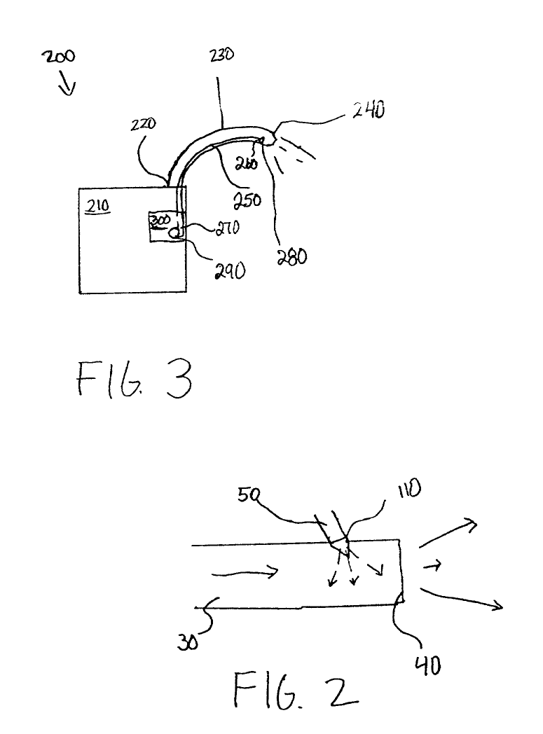

Referring to FIGS. 1 and 2 in combination, there is shown an air delivery system 10. An air delivery unit 15 is provided to deliver air or other gases through an outlet 20. A generally tubular delivery tube 30 is connected to the outlet 20 for delivering air from the system 10 to a desired location. In certain preferred embodiments, the delivery tube 30 is constructed of a resilient material to allow a user to direct the airflow to a desired location. The delivery tube 30 terminates at a delivery outlet 40, which in certain embodiments may be tapered to aid in directing the airflow.

Referring still to FIGS. 1 and 2, a misting tube 50 is provided having a fluid receiving end 60 and a fluid delivering end 70. The fluid delivering end 70 is connected to the delivery tube 30 at a point adjacent to the delivery outlet 40, best seen in FIG. 2. At the fluid receiving end 60 of the misting tube 50, a fluid source 80 is connected thereto for providing fluid 90 through the misting tube 100. The fluid source 80 may include a pump 100, such as a diaphragm pump and the like, for moving fluid 90 from the fluid source 80 through the misting tube 50. In this embodiment, the fluid source 80 is externally located from the air delivery system 10. The fluid 90 may comprise any substance, but preferably comprises a medicated skin lubricant such as lanolin and the like. Importantly, the fluid 90 may be fragranced or otherwise treated so as to affect the surface of the desired location with the desired property. It is also contemplated that the fluid 90 may have combinations of desired properties, depending upon the needs of the user.

In a preferred embodiment, the fluid delivering end 70 of the misting tube 50 comprises a nozzle 110. The nozzle 110 functions to deliver fluid 90 in a misting pattern into the delivery tube 30. Once the fluid 90 has been delivered into the delivery tube 30, it combines with air flowing through the delivery tube 30. The resulting air (or other gas)/fluid mixture may then be delivered onto a desired location. The nozzle 110 may be set at a predetermined angle with respect to the direction of air flowing through the delivery tube 30 to maximize the efficiency of the misting pattern. The nozzle 110 may also be an adjustable nozzle, such that modification of the nozzle 110 will adjust the resulting misting pattern. In some embodiments, the nozzle 110 may be removed and replaced with other nozzles having different properties, such as those having increased flow rates and alternate misting pattern properties. In these cases, the fluid delivering end 70 of the misting tube 50 may be provided with threads adapted to mate with nozzles having predetermined connection characteristics. Other conventional seaming means may be used. It is contemplated that the misting tube 50 may be made of a resilient material, such as when the fluid source 90 is located at a distance from the air delivery system 10.

While it is appreciated that introduction of the fluid 90 into the delivery tube 30 and the resulting combination of the fluid 90 with air flowing through the delivery tube 30 may have a cooling and/or heating effect at the delivery outlet 40, more cooling may be required. Thus, in certain embodiments, the air delivery system 10 may incorporate an air conditioning unit or other system which assists in cooling and/or heating the air.

In operation, the air delivery system 10 is activated to allow air to flow through the delivery tube 30. Fluid 90 from the misting source is introduced in-stream with the air flow through the nozzle 110, which introduces the fluid 90 in a misting pattern. The combined fluid/air may then be directed through the delivery outlet 40 at a desired location. If necessary, the air from the air delivery system 10 may be cooled and/or heated using the aforementioned techniques. Because the fluid may be medicated, fragranced or otherwise treated, a specifically tailored air/fluid mixture may be applied in a specific treatment, depending on the needs of the user.

It is especially appreciated that the present invention may be used in dermatological treatments utilizing laser or light. In such treatments, the location where the laser or other method of treating is applied will often emit an undesirable odor. The present invention eliminates this odor by providing a pleasing fragrance at the location of the treatment. In addition, the present invention provides for medicated fluid 90 to be applied at the treatment site, thus improving the recovery time of the person being treated. The fluid 90 may also be lubricated to improve the comfort of the person being treated. Because extraneous materials are minimized, the present invention provides an efficient and economical alternative to those of the prior art.

Referring now to FIG. 3, there is shown an air or other gas delivery system 200 in an alternate embodiment. The air delivery system 200 includes an air or other gas delivery unit 210 which delivers air through an air outlet 220. A delivery tube 230 connects to the air outlet 220 and terminates at a delivery outlet 240. As in prior embodiments, the delivery tube 230 may be constructed of resilient material. A misting tube 250 is provided within the delivery tube 230 and has a fluid delivering end 260 and a fluid receiving end 270. The fluid delivering end 260 terminates at a nozzle 280, which is positioned to direct flow from the misting tube 250 into air flowing through the delivery tube 230. In this embodiment, the fluid receiving end 270 is fluidly connected to a fluid source outlet 290, which is connected to a fluid source 300 internally located within the air delivery unit 210. It is appreciated that the fluid source 300 may, as in prior embodiments, be located externally from the air delivery unit 210. The air delivery system 200 functions in the same manner as described in the prior embodiments.

It is believed that the operation and construction of the present invention will be apparent from the foregoing description of the preferred exemplary embodiments. While the jet cooling and/or heating apparatus and method shown is described as being preferred, it will be obvious to a person of ordinary skill in the art that various changes and modifications made may be made therein without departing from the spirit and the scope of the invention.

What is claimed is:

1. A fluid delivery apparatus, comprising:
an air delivery system having an air outlet, said air delivery system delivering air through said air outlet;
a resilient delivery tube connected to said air outlet, said resilient delivery tube terminating at a delivery outlet;
a misting tube having a fluid receiving end and a fluid delivering end, said fluid delivering end being connected to said resilient delivery tube at a point adjacent to said delivery outlet;
a fluid source connected to said misting tube at said fluid receiving end;
wherein fluid from said fluid source is combined in-stream in a misting pattern with air from said resilient delivery tube to provide said fluid at a predetermined location.

2. The fluid delivery apparatus of claim 1, wherein said misting tube has a mist nozzle for forming said misting pattern, said mist nozzle being connected to said resilient delivery tube at a point adjacent to said delivery outlet.

3. The fluid delivery apparatus of claim 2, wherein said mist nozzle is fixed at a predetermined angle with respect to air flow through said resilient delivery tube.

4. The fluid delivery apparatus of claim 2, wherein said mist nozzle is adjustable to form an angle with respect to air flow through said resilient delivery tube.

5. The fluid delivery apparatus of claim 2, wherein said misting pattern is adjustable.

6. The fluid delivery apparatus of claim 1, wherein fluid from said fluid source is fragranced.

7. The fluid delivery apparatus of claim 1 wherein fluid from said fluid source is medicated.

8. The fluid delivery apparatus of claim 1, wherein air from said air delivery system is temperature adjusted prior to combination with said fluid in said resilient delivery tube.

9. The fluid delivery apparatus of claim 1, wherein fluid from said fluid source is a lubricant.

10. The fluid delivery apparatus of claim 1, wherein said fluid source is located within said air delivery system.

11. The fluid delivery apparatus of claim 10, wherein said misting tube is located within said resilient delivery tube.

12. A fluid delivery apparatus, comprising:
an air delivery system having an air outlet, said air delivery system delivering air through said air outlet;
a generally tubular resilient delivery tube connected to said air outlet, said generally tubular resilient delivery tube terminating at a delivery outlet;
a misting tube having a fluid receiving end and a fluid delivering end, said fluid delivering end having an adjustable nozzle connected thereto, said adjustable nozzle being connected to said generally tubular resilient delivery tube at a point adjacent to said delivery outlet;
a fragranced fluid source connected to said misting tube at said fluid receiving end;
wherein fragranced fluid from said fluid source is introduced through said adjustable nozzle in-stream in a misting pattern with air flowing from said resilient delivery tube to provide said fragranced fluid at a predetermined location.

13. A method of delivering fluid, comprising the steps of:
providing a first gaseous fluid supply system for delivering said gaseous fluid through a delivery tube having a delivery outlet to a desired location;
introducing a second fluid from a fluid source in a misting pattern into said delivery tube near said delivery outlet;
directing flow of said first gaseous fluid and said second fluid from said delivery tube to a desired location.

14. The method of claim 13, wherein said first gaseous fluid from said first gaseous fluid supply system is temperature adjusted.

15. The method of claim 14, wherein said second fluid is fragranced.

16. The method of claim 14, wherein said second fluid is medicated.

17. The method of claim 14, wherein said second fluid is a lubricant.

18. The method of claim 14, wherein said first gaseous fluid is cooled.

19. The method of claim 14, wherein said first gaseous fluid is heated.
